SEQUENCE LISTING (33 sequences)

SEQ ID NO: 1 - DNA, 56 bp, synthetic construct
  source 1..56: mol_type = other DNA
cgacgaacggtcgctcgacgtacgtcgacctgaaaaggtcgacgtcgctcgacgta

SEQ ID NO: 2 - DNA, 56 bp, synthetic construct
  source 1..56: mol_type = other DNA
cgacgaacggtcgctcgacgtacgtcgcctagaaataggcgacgtcgctcgacgta

SEQ ID NO: 3 - DNA, 56 bp, synthetic construct
  source 1..56: mol_type = other DNA
cgacgaacggtcgctcgacgtacgtccctgagaaatcagggacgtcgctcgacgta

SEQ ID NO: 4 - DNA, 56 bp, synthetic construct
  source 1..56: mol_type = other DNA
cgacgaacggtcgctcgacgtacgtcctcgagaaatcgaggacgtcgctcgacgta

SEQ ID NO: 5 - DNA, 56 bp, synthetic construct
  source 1..56: mol_type = other DNA
cgacgaacggtcgctcgacgtacgccttcgagaaatcgaaggcgtcgctcgacgta

SEQ ID NO: 6 - DNA, 56 bp, synthetic construct
  source 1..56: mol_type = other DNA
cgacgaacggtcgctcgacgtagcctctcgagaaatcgagaggctcgctcgacgta

SEQ ID NO: 7 - DNA, 56 bp, synthetic construct
  source 1..56: mol_type = other DNA
aaaaaaaaaaaaaaaaaaaaaacgtccctgagaaatcagggacgtcgctcgacgta

SEQ ID NO: 8 - DNA, 56 bp, synthetic construct
  source 1..56: mol_type = other DNA
ttttttttttttttttttttttcgtccctgagaaatcagggacgtcgctcgacgta

SEQ ID NO: 9 - DNA, 52 bp, synthetic construct
  source 1..52: mol_type = other DNA
gaacggtcgctcgacgtacgtccctgagaaatcagggacgtcgctcgacgta

SEQ ID NO: 10 - DNA, 48 bp, synthetic construct
  source 1..48: mol_type = other DNA
ggtcgctcgacgtacgtccctgagaaatcagggacgtcgctcgacgta

SEQ ID NO: 11 - DNA, 44 bp, synthetic construct
  source 1..44: mol_type = other DNA
gctcgacgtacgtccctgagaaatcagggacgtcgctcgacgta

SEQ ID NO: 12 - DNA, 35 bp, synthetic construct
  source 1..35: mol_type = other DNA
acgtcgacctgaaaaggtcgacgtcgctcgacgta

SEQ ID NO: 13 - DNA, 38 bp, synthetic construct
  source 1..38: mol_type = other DNA
cgtacgtcgacctgaaaaggtcgacgtcgctcgacgta

SEQ ID NO: 14 - DNA, 41 bp, synthetic construct
  source 1..41: mol_type = other DNA
cgacgtacgtcgacctgaaaaggtcgacgtcgctcgacgta

SEQ ID NO: 15 - DNA, 44 bp, synthetic construct
  source 1..44: mol_type = other DNA
gctcgacgtacgtcgacctgaaaaggtcgacgtcgctcgacgta

SEQ ID NO: 16 - DNA, 47 bp, synthetic construct
  source 1..47: mol_type = other DNA
gtcgctcgacgtacgtcgacctgaaaaggtcgacgtcgctcgacgta

SEQ ID NO: 17 - DNA, 50 bp, synthetic construct
  source 1..50: mol_type = other DNA
acggtcgctcgacgtacgtcgacctgaaaaggtcgacgtcgctcgacgta

SEQ ID NO: 18 - DNA, 53 bp, synthetic construct
  source 1..53: mol_type = other DNA
cgaacggtcgctcgacgtacgtcgacctgaaaaggtcgacgtcgctcgacgta

SEQ ID NO: 19 - DNA, 56 bp, synthetic construct; identical to SEQ ID NO: 1
  source 1..56: mol_type = other DNA

SEQ ID NO: 20 - DNA, 21 bp, synthetic construct
  source 1..21: mol_type = other DNA
  modified_base 1: mod_base = OTHER; mod_base = OTHER; note = uracil
  modified_base 5: mod_base = OTHER; mod_base = OTHER; note = uracil
  modified_base 21: mod_base = OTHER; mod_base = OTHER; note = uracil
tacgtcgagcgacgtcgacct

SEQ ID NO: 21 - DNA, 35 bp, synthetic construct; identical to SEQ ID NO: 12
  source 1..35: mol_type = other DNA

SEQ ID NO: 22 - DNA, 38 bp, synthetic construct; identical to SEQ ID NO: 13
  source 1..38: mol_type = other DNA

SEQ ID NO: 23 - DNA, 41 bp, synthetic construct; identical to SEQ ID NO: 14
  source 1..41: mol_type = other DNA

SEQ ID NO: 24 - DNA, 44 bp, synthetic construct; identical to SEQ ID NO: 15
  source 1..44: mol_type = other DNA

SEQ ID NO: 25 - DNA, 47 bp, synthetic construct; identical to SEQ ID NO: 16
  source 1..47: mol_type = other DNA

SEQ ID NO: 26 - DNA, 50 bp, synthetic construct; identical to SEQ ID NO: 17
  source 1..50: mol_type = other DNA

SEQ ID NO: 27 - DNA, 53 bp, synthetic construct; identical to SEQ ID NO: 18
  source 1..53: mol_type = other DNA

SEQ ID NO: 28 - DNA, 56 bp, synthetic construct; identical to SEQ ID NO: 1
  source 1..56: mol_type = other DNA

SEQ ID NO: 29 - DNA, 83 bp, synthetic construct
  misc_feature 12: note = RNA
  misc_feature 14: note = RNA
  misc_feature 15: note = RNA
  misc_feature 22: note = RNA
  misc_feature 23: note = RNA
  misc_feature 24: note = RNA
  misc_feature 25: note = RNA
  misc_feature 31: note = RNA
  misc_feature 33: note = RNA
  misc_feature 36: note = RNA
  misc_feature 38: note = RNA
  misc_feature 41: note = RNA
  misc_feature 39: note = RNA
  misc_feature 42: note = RNA
  misc_feature 72: note = RNA
  misc_feature 76: note = RNA
  misc_feature 82: note = RNA
  source 1..83: mol_type = other DNA
  modified_base 12: mod_base = OTHER; mod_base = OTHER; note = uracil
  modified_base 14: mod_base = OTHER; mod_base = OTHER; note = uracil
  modified_base 15: mod_base = OTHER; mod_base = OTHER; note = uracil
  modified_base 22: mod_base = OTHER; mod_base = OTHER; note = uracil
  modified_base 23: mod_base = OTHER; mod_base = OTHER; note = uracil
  modified_base 24: mod_base = OTHER; mod_base = OTHER; note = uracil
  modified_base 25: mod_base = OTHER; mod_base = OTHER; note = uracil
  modified_base 31: mod_base = OTHER; mod_base = OTHER; note = uracil
  modified_base 33: mod_base = OTHER; mod_base = OTHER; note = uracil
  modified_base 36: mod_base = OTHER; mod_base = OTHER; note = uracil
  modified_base 38: mod_base = OTHER; mod_base = OTHER; note = uracil
  modified_base 39: mod_base = OTHER; mod_base = OTHER; note = uracil
  modified_base 40: mod_base = OTHER; mod_base = OTHER; note = uracil
  modified_base 41: mod_base = OTHER; mod_base = OTHER; note = uracil
  modified_base 72: mod_base = OTHER; mod_base = OTHER; note = uracil
  modified_base 76: mod_base = OTHER; mod_base = OTHER; note = uracil
  modified_base 82: mod_base = OTHER; mod_base = OTHER; note = uracil
gggcgaggagctgttcaccggttttagagctatgctgttttgcgacgtacgtcgacctgaaaaggtcgacgtcgctcgacgta

SEQ ID NO: 30 - DNA, 22 bp, synthetic construct
  source 1..22: mol_type = other DNA
  modified_base 1: mod_base = OTHER; mod_base = OTHER; note = uracil
  modified_base 5: mod_base = OTHER; mod_base = OTHER; note = uracil
  modified_base 15: mod_base = OTHER; mod_base = OTHER; note = uracil
  modified_base 20: mod_base = OTHER; mod_base = OTHER; note = uracil
  modified_base 21: mod_base = OTHER; mod_base = OTHER; note = uracil
  modified_base 22: mod_base = OTHER; mod_base = OTHER; note = uracil
tacgtcgagcgacgtcgacttt

SEQ ID NO: 31 - DNA, 21 bp, synthetic construct
  misc_feature 1: note = RNA
  misc_feature 5: note = RNA
  misc_feature 21: note = RNA
  source 1..21: mol_type = other DNA
  modified_base 1: mod_base = OTHER; mod_base = OTHER; note = 2'-O-methyl modification
  modified_base 2: mod_base = OTHER; mod_base = OTHER; note = 2'-O-methyl modification
  modified_base 5: mod_base = OTHER; mod_base = OTHER; note = 2'-O-methyl modification
  modified_base 8: mod_base = OTHER; mod_base = OTHER; note = 2'-O-methyl modification
  modified_base 11: mod_base = OTHER; mod_base = OTHER; note = 2'-O-methyl modification
  modified_base 14: mod_base = OTHER; mod_base = OTHER; note = 2'-O-methyl modification
  modified_base 17: mod_base = OTHER; mod_base = OTHER; note = 2'-O-methyl modification
  modified_base 20: mod_base = OTHER; mod_base = OTHER; note = 2'-O-methyl modification
  modified_base 21: mod_base = OTHER; mod_base = OTHER; note = 2'-O-methyl modification
  modified_base 1: mod_base = OTHER; mod_base = OTHER; note = 2'-O-methyl modification
  modified_base 1: mod_base = OTHER; mod_base = OTHER; note = uracil
  modified_base 5: mod_base = OTHER; mod_base = OTHER; note = uracil
  modified_base 11: mod_base = OTHER; mod_base = OTHER; note = uracil
tacgtcgagcgacgtcgacct

SEQ ID NO: 32 - DNA, 83 bp, synthetic construct; identical to SEQ ID NO: 29
  misc_feature 12: note = RNA
  misc_feature 14: note = RNA
  misc_feature 15: note = RNA
  misc_feature 22: note = RNA
  misc_feature 23: note = RNA
  misc_feature 24: note = RNA
  misc_feature 25: note = RNA
  misc_feature 31: note = RNA
  misc_feature 33: note = RNA
  misc_feature 36: note = RNA
  misc_feature 38: note = RNA
  misc_feature 39: note = RNA
  misc_feature 40: note = RNA
  misc_feature 41: note = RNA
  misc_feature 72: note = RNA
  misc_feature 76: note = RNA
  misc_feature 82: note = RNA
  source 1..83: mol_type = other DNA
  modified_base 2: mod_base = OTHER; mod_base = OTHER; note = 2'-O-methyl modification
  modified_base 40: mod_base = OTHER; mod_base = OTHER; note = 2'-O-methyl modification
  modified_base 1: mod_base = OTHER; mod_base = OTHER; note = 2'-O-methyl modification
  modified_base 41: mod_base = OTHER; mod_base = OTHER; note = 2'-O-methyl modification
  modified_base 42: mod_base = OTHER; mod_base = OTHER; note = 2'-O-methyl modification
  modified_base 50: mod_base = OTHER; mod_base = OTHER; note = 2'-O-methyl modification
  modified_base 72: mod_base = OTHER; mod_base = OTHER; note = 2'-O-methyl modification
  modified_base 73: mod_base = OTHER; mod_base = OTHER; note = 2'-O-methyl modification
  modified_base 74: mod_base = OTHER; mod_base = OTHER; note = 2'-O-methyl modification
  modified_base 75: mod_base = OTHER; mod_base = OTHER; note = 2'-O-methyl modification
  modified_base 76: mod_base = OTHER; mod_base = OTHER; note = 2'-O-methyl modification
  modified_base 77: mod_base = OTHER; mod_base = OTHER; note = 2'-O-methyl modification
  modified_base 78: mod_base = OTHER; mod_base = OTHER; note = 2'-O-methyl modification
  modified_base 78: mod_base = OTHER; mod_base = OTHER; note = 2'-O-methyl modification
  modified_base 79: mod_base = OTHER; mod_base = OTHER; note = 2'-O-methyl modification
  modified_base 81: mod_base = OTHER; mod_base = OTHER; note = 2'-O-methyl modification
  modified_base 82: mod_base = OTHER; mod_base = OTHER; note = 2'-O-methyl modification
  modified_base 83: mod_base = OTHER; mod_base = OTHER; note = 2'-O-methyl modification
  modified_base 2: mod_base = OTHER; mod_base = OTHER; note = contains phosphorothioate bond
  modified_base 3: mod_base = OTHER; mod_base = OTHER; note = contains phosphorothioate bond
  modified_base 42: mod_base = OTHER; mod_base = OTHER; note = contains phosphorothioate bond
  modified_base 43: mod_base = OTHER; mod_base = OTHER; note = contains phosphorothioate bond
  modified_base 44: mod_base = OTHER; mod_base = OTHER; note = contains phosphorothioate bond
  modified_base 45: mod_base = OTHER; mod_base = OTHER; note = contains phosphorothioate bond
  modified_base 46: mod_base = OTHER; mod_base = OTHER; note = contains phosphorothioate bond
  modified_base 47: mod_base = OTHER; mod_base = OTHER; note = contains phosphorothioate bond
  modified_base 48: mod_base = OTHER; mod_base = OTHER; note = contains phosphorothioate bond
  modified_base 49: mod_base = OTHER; mod_base = OTHER; note = contains phosphorothioate bond
  modified_base 50: mod_base = OTHER; mod_base = OTHER; note = contains phosphorothioate bond
  modified_base 60: mod_base = OTHER; mod_base = OTHER; note = contains phosphorothioate bond
  modified_base 61: mod_base = OTHER; mod_base = OTHER; note = contains phosphorothioate bond
  modified_base 62: mod_base = OTHER; mod_base = OTHER; note = contains phosphorothioate bond
  modified_base 82: mod_base = OTHER; mod_base = OTHER; note = contains phosphorothioate bond
  modified_base 83: mod_base = OTHER; mod_base = OTHER; note = contains phosphorothioate bond
  misc_feature 1: note = RNA
  misc_feature 2: note = RNA
  misc_feature 3: note = RNA
  misc_feature 4: note = RNA
  misc_feature 5: note = RNA
  misc_feature 6: note = RNA
  misc_feature 7: note = RNA
  misc_feature 8: note = RNA
  misc_feature 9: note = RNA
  misc_feature 10: note = RNA
  misc_feature 11: note = RNA
  misc_feature 13: note = RNA
  misc_feature 16: note = RNA
  misc_feature 17: note = RNA
  misc_feature 18: note = RNA
  misc_feature 19: note = RNA
  misc_feature 20: note = RNA
  misc_feature 21: note = RNA
  misc_feature 26: note = RNA
  misc_feature 27: note = RNA
  misc_feature 28: note = RNA
  misc_feature 29: note = RNA
  misc_feature 30: note = RNA
  misc_feature 32: note = RNA
  misc_feature 34: note = RNA
  misc_feature 37: note = RNA
  misc_feature 35: note = RNA
  modified_base 12: mod_base = OTHER; mod_base = OTHER; note = uracil
  modified_base 14: mod_base = OTHER; mod_base = OTHER; note = uracil
  modified_base 15: mod_base = OTHER; mod_base = OTHER; note = uracil
  modified_base 22: mod_base = OTHER; mod_base = OTHER; note = uracil
  modified_base 23: mod_base = OTHER; mod_base = OTHER; note = uracil
  modified_base 24: mod_base = OTHER; mod_base = OTHER; note = uracil
  modified_base 25: mod_base = OTHER; mod_base = OTHER; note = uracil
  modified_base 31: mod_base = OTHER; mod_base = OTHER; note = uracil
  modified_base 33: mod_base = OTHER; mod_base = OTHER; note = uracil
  modified_base 36: mod_base = OTHER; mod_base = OTHER; note = uracil
  modified_base 38: mod_base = OTHER; mod_base = OTHER; note = uracil
  modified_base 39: mod_base = OTHER; mod_base = OTHER; note = uracil
  modified_base 40: mod_base = OTHER; mod_base = OTHER; note = uracil
  modified_base 41: mod_base = OTHER; mod_base = OTHER; note = uracil
  modified_base 72: mod_base = OTHER; mod_base = OTHER; note = uracil
  modified_base 76: mod_base = OTHER; mod_base = OTHER; note = uracil
  modified_base 82: mod_base = OTHER; mod_base = OTHER; note = uracil

SEQ ID NO: 33 - DNA, 22 bp, synthetic construct
  misc_feature 1: note = RNA
  misc_feature 5: note = RNA
  misc_feature 15: note = RNA
  misc_feature 20: note = RNA
  misc_feature 21: note = RNA
  misc_feature 22: note = RNA
  source 1..22: mol_type = other DNA
  modified_base 1: mod_base = OTHER; mod_base = OTHER; note = 2'-methoxyethyl modification
  modified_base 2: mod_base = OTHER; mod_base = OTHER; note = 2'-methoxyethyl modification
  modified_base 3: mod_base = OTHER; mod_base = OTHER; note = 2'-methoxyethyl modification
  modified_base 4: mod_base = OTHER; mod_base = OTHER; note = 2'-methoxyethyl modification
  modified_base 6: mod_base = OTHER; mod_base = OTHER; note = 2'-methoxyethyl modification
  modified_base 7: mod_base = OTHER; mod_base = OTHER; note = 2'-methoxyethyl modification
  modified_base 8: mod_base = OTHER; mod_base = OTHER; note = 2'-methoxyethyl modification
  modified_base 10: mod_base = OTHER; mod_base = OTHER; note = 2'-methoxyethyl modification
  modified_base 11: mod_base = OTHER; mod_base = OTHER; note = 2'-methoxyethyl modification
  modified_base 12: mod_base = OTHER; mod_base = OTHER; note = 2'-methoxyethyl modification
  modified_base 14: mod_base = OTHER; mod_base = OTHER; note = 2'-methoxyethyl modification
  modified_base 15: mod_base = OTHER; mod_base = OTHER; note = 2'-methoxyethyl modification
  modified_base 16: mod_base = OTHER; mod_base = OTHER; note = 2'-methoxyethyl modification
  modified_base 18: mod_base = OTHER; mod_base = OTHER; note = 2'-methoxyethyl modification
  modified_base 19: mod_base = OTHER; mod_base = OTHER; note = 2'-methoxyethyl modification
  modified_base 20: mod_base = OTHER; mod_base = OTHER; note = 2'-methoxyethyl modification
  modified_base 22: mod_base = OTHER; mod_base = OTHER; note = 2'-methoxyethyl modification
  modified_base 5: mod_base = OTHER; mod_base = OTHER; note = locked nucleic acid modification
  modified_base 9: mod_base = OTHER; mod_base = OTHER; note = locked nucleic acid modification
  modified_base 13: mod_base = OTHER; mod_base = OTHER; note = locked nucleic acid modification
  modified_base 17: mod_base = OTHER; mod_base = OTHER; note = locked nucleic acid modification
  modified_base 21: mod_base = OTHER; mod_base = OTHER; note = locked nucleic acid modification
  modified_base 1: mod_base = OTHER; mod_base = OTHER; note = uracil
  modified_base 5: mod_base = OTHER; mod_base = OTHER; note = uracil
  modified_base 15: mod_base = OTHER; mod_base = OTHER; note = uracil
  modified_base 20: mod_base = OTHER; mod_base = OTHER; note = uracil
  modified_base 21: mod_base = OTHER; mod_base = OTHER; note = uracil
  modified_base 22: mod_base = OTHER; mod_base = OTHER; note = uracil
tacgtcgagcgacgtcgacttt

(19) United States
(12) Patent Application Publication
(10) Pub. No.: US 2026/0103702 A1
(43) Pub. Date: Apr. 16, 2026

(54) COMPOSITIONS AND METHODS FOR CRISPR MODULATION

(71) Applicant: Board of Trustees of Southern Illinois University, Carbondale, IL (US)

(72) Inventors: Keith Gagnon, Pfafftown, NC (US); Seth Eddington, Springfield, IL (US); Christopher Barkau, Carbondale, IL (US)

(21) Appl. No.: 19/117,193

(22) PCT Filed: Sep. 29, 2023
(86) PCT No.: PCT/US2023/075654

§ 371 Date: Mar. 31, 2025

Related U.S. Application Data
(60) Provisional application No. 63/378,038, filed on Sep. 30, 2022.

(51) Int. Cl. C12N 15/11; C12N 9/22

(52) U.S. Cl. CPC C12N 15/11; C12N 9/226; C12N 2310/20

(57) ABSTRACT

A composition, a method for selectively altering expression of a gene, and a method of selectively modulating activity of a Cas protein is provided. The composition includes a nucleotide regulator wherein the regulator is an oligonucleotide sequence at least 95% identical to a sequence selected from the group consisting of SEQ ID NOs. 1-6 and 8-19 or a functional fragment thereof and may comprise a derepressor wherein the derepressor has an oligonucleotide sequence at least 95% identical to SEQ. ID. NO. 20 or a functional fragment thereof. The method of selectively altering gene expression includes administering the composition containing a nucleotide regulator. The method of selectively modulating activity of a Cas protein includes administering a composition of a repressor and a derepressor to a subject, wherein the repressor competes with or disrupts the PM module structure of the Cas protein, and the derepressor reverses the disruption of the repressor.

### CROSS-REFERENCE TO RELATED APPLICATIONS

This application claims priority to U.S. Provisional Patent Application No. 63/378,038, filed Sep. 30, 2022.

### STATEMENT REGARDING FEDERALLY SPONSORED RESEARCH OR DEVELOPMENT

This invention was made with government support under grant number GM135646, awarded by the National Institutes of Health. The government has certain rights in the invention.

### REFERENCE TO SEQUENCE LISTING

The instant application contains a Sequence Listing that has been submitted electronically in .XML format and is hereby incorporated by reference in its entirety. The Sequence Listing: is named “50097.xml”, is 140 kilobytes in size, and was created on 9/29/2023.

### FIELD OF THE INVENTION

The present disclosure relates to compositions and methods for modulating gene expression via CRISPR enzymes.

### BACKGROUND OF THE INVENTION

Clustered regularly interspaced short palindromic repeats (CRISPR) and associated Cas (CRISPR-associated) proteins constitute the CRISPR-Cas system. This system originated in bacteria as a defense mechanism against bacteriophages. Cas enzymes are DNA endonucleases, which utilize a RNA dual guide system in order to specifically target and cleave DNA in a sequence-dependent manner. The dual guide system is comprised of a CRISPR RNA (crRNA) which is bound to a trans-activated crRNA (tracrRNA).

Researchers have attempted to adapt the CRISPR-Cas systems (“CRISPR”) to aid in treatment of genetic diseases, an approach now at the forefront of potential gene therapy strategies. CRISPR has potential applications in treating genetic diseases, diagnostics and improving the properties of agricultural cultivars, but research into these potential applications remains in early stages of development. Such early-stage research hypothesizes that, for example, CRISPR-Cas9 technology can be custom-programmed to target specific genetic sequences at precise predetermined locations. This proposed, programmability may allow scientists use CRISPR systems as effective sequence-specific genome editing tools.

While CRISPR systems promise to positively impact gene-targeted therapeutics, there are still major concerns surrounding specificity and the potential to introduce unwanted and irreversible genetic mutations, known as off-target effects. Non-specific genome editing remains as one of the most critical barriers to clinical applications. To generate safe CRISPR-based medicines, off-target effects need to be controlled via regulation of the CRISPR compositions and systems. Thus, there is a need for CRISPR compositions and systems that are regulatable and switchable.

### SUMMARY

In one aspect, a composition is provided comprising a nucleotide regulator, wherein the nucleotide regulator is an oligonucleotide sequence at least 95% identical to a sequence selected from the group consisting of SEQ. ID. NOs. 1-6 and 8-19 or a functional fragment thereof.

In some embodiments, the composition includes an oligonucleotide sequence comprising a linker or spacer of about 0- about 50 nucleotides in length.

In other embodiments, the composition further comprises a Cas protein.

In yet another embodiment, the composition comprises a tracrRNA and a Cas protein where the Cas protein comprises Cas9, or dead Cas9 (dCas9).

In some embodiments, the composition includes a derepressor.

In other embodiments, the composition includes a derepressor that is a derepressor oligonucleotide.

In yet another embodiment, the composition comprises a derepressor to nucleotide regulator ratio from about 1:1 to about 1:20.

In some embodiments, the composition includes a derepressor oligonucleotide which is an oligonucleotide sequence at least 95% identical to SEQ. ID. NO. 20 or a functional fragment thereof.

In another aspect, a method for selectively altering expression of at least one gene product is provided wherein the method includes administering the composition to a subject.

In other embodiments, the method includes administering the composition wherein the composition further comprises an oligonucleotide sequence comprising a linker or spacer of about 0- about 50 nucleotides in length.

In yet another embodiment, the method includes administering a composition wherein the composition further comprises a Cas protein.

In some embodiments, the method includes administering a composition wherein the composition further comprises a tracrRNA and a Cas protein wherein the Cas protein comprises Cas9, or dead Cas9 (dCas9).

In other embodiments, the method includes administering a composition wherein the composition further includes a derepressor.

In yet another embodiment, the method includes administering a composition wherein the composition further includes a derepressor oligonucleotide.

In some embodiments, the method includes administering a composition wherein the composition has a derepressor to nucleotide regulator ratio from about 1:1 to about 1:20.

In other embodiments, the method includes administering a composition wherein the composition has a derepressor wherein the derepressor is at least 95% identical to SEQ. ID. NO. 20 or a functional fragment thereof.

In another aspect, a method of selectively modulating the activity of a Cas protein is provided wherein the method includes administering a composition comprising a repressor and a derepressor to a subject, wherein the repressor competes with or disrupts the PM module structure of the Cas protein, and wherein the derepressor reverses the disruption of the repressor.

In yet another embodiment, the method includes administering a composition wherein the repressor comprises a nucleotide regulator, wherein the nucleotide regulator is at least 95% identical to a sequence selected from the group consisting of SEQ. ID. NOs. 1-6 and 8-19 or a functional fragment thereof.

In some embodiments, the method includes administering a composition wherein the derepressor comprises an oligonucleotide sequence at least 95% identical to SEQ ID 20 or a functional fragment thereof.

In other embodiments, the method includes administering a composition wherein the repressor and derepressor are included in a liposome.

### DESCRIPTION OF THE DRAWINGS

FIG. 1 is an illustration of cis-acting switchable nucleic acid-based regulators (cis-SNuBRs) of CRISPR-Cas9 without a derepressor:

FIG. 2 is a graph showing the inhibition of in vitro cleavage of target substrate DNA by Cas9 for the various DNA oligonucleotides SEQ. ID NOs. 1-12:

FIG. 3 is a graph showing the inhibition of in vitro cleavage of target substrate DNA by Cas9 for the various DNA oligonucleotides SEQ. ID NOs. 21-28;

FIG. 4 is an illustration of the mechanism of the modulatory action of cis-acting switchable nucleic acid-based regulators (cis-SNuBRs) of CRISPR-Cas9 with and without a derepressor;

FIG. 5 is a graph showing the in vitro cleavage of Cas9 in the presence of repressor spacer variant oligonucleotides and a derepressor;

FIG. 6 is a graph showing the in vitro cleavage activity of Cas9 in in the presence of repressor oligonucleotides and a derepressor with varying derepressor incubation times:

FIG. 7 is a bar graph showing the in vitro cleavage activity of Cas9 in in the presence of repressor oligonucleotides and a derepressor with varying concentrations.

FIG. 8 is the precent edited gene target by Cas9 in the presence of the repressor oligonucleotide SEQ. NO. 29 and the derepressor SEQ. ID. NO. 30.

FIG. 9 is an oligonucleotide table that sets forth sequences of certain oligonucleotides used in various embodiments of the disclosure.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The following discussion is presented to enable a person skilled in the art to make and use embodiments of the disclosure. Various modifications to the illustrated embodiments will be readily apparent to those skilled in the art, and the generic principles herein can be applied to other embodiments and applications without departing from embodiments of the disclosure. Thus, embodiments of the disclosure are not intended to be limited to embodiments shown but are to be accorded the widest scope consistent with the principles and features disclosed herein. The following detailed description is to be read with reference to the figures, in which like elements in different figures have like reference numerals. The figures depict selected embodiments and are not intended to limit the scope of embodiments of the disclosure. Skilled artisans will recognize the examples provided herein have many useful alteratives and fall within the scope of embodiments of the disclosure.

According to the teachings therein, the discovery of cis-acting switchable nucleic acid-based regulators (cis-SNuBRs) for, e.g., switchable Cas9 activity is provided. In the default state, cis-SNuBRs inhibit Cas9 and through the introduction of a “derepressor” oligo or the presence of a selected nucleic acid, may release inhibition and activate Cas9 DNA cleavage activity. In some embodiments, the cis-SNuBRs may regulate Cas (e.g., Cas9) activity. The design of cis-SNuBRs may allow for controlled activation of Cas enzymes temporally as well as possibly in a tissue or cell specific manner. The cis-SNuBRs characteristic switchability may reduce sequence and tissue specific off-target effects and enable safer use in clinical and research settings.

According to the teachings herein, cis-SNuBRs have been shown to effectively inhibit the catalytic activity of the Cas (e.g., Cas9) enzyme in vitro and after introduction of a derepressor oligo, cis-SNuBRs reactivate the catalytic activity.

The following drawings form part of the present specification and are included to further demonstrate certain aspects of the present invention. The invention may be better understood by reference to one or more of these drawings in combination with the detailed description of specific embodiments presented herein. However, those of skill in the art will understand that the drawings, described below, are for illustrative purposes only. The drawings are not intended to limit the scope of the present teachings in any way.

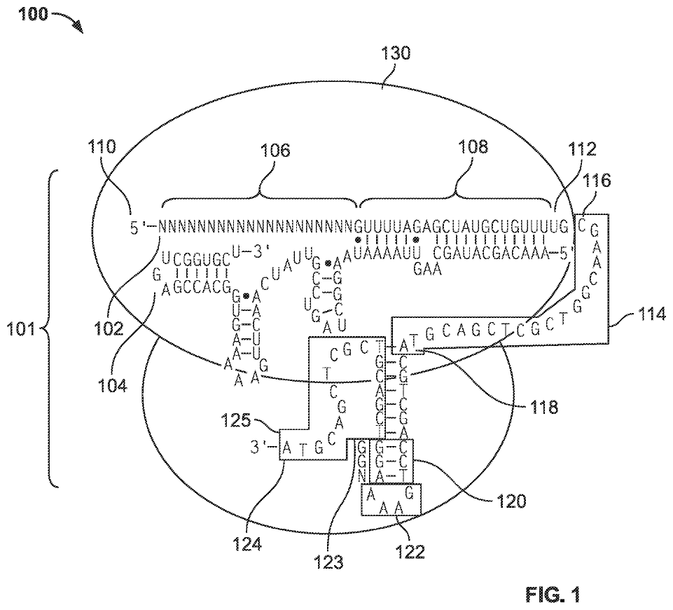

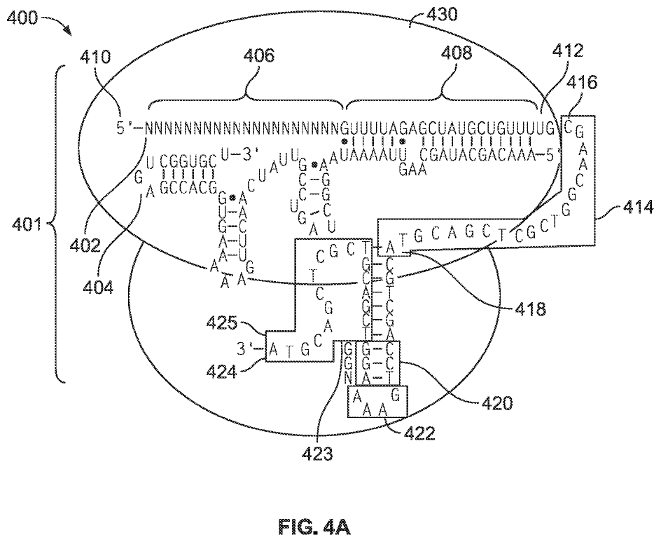

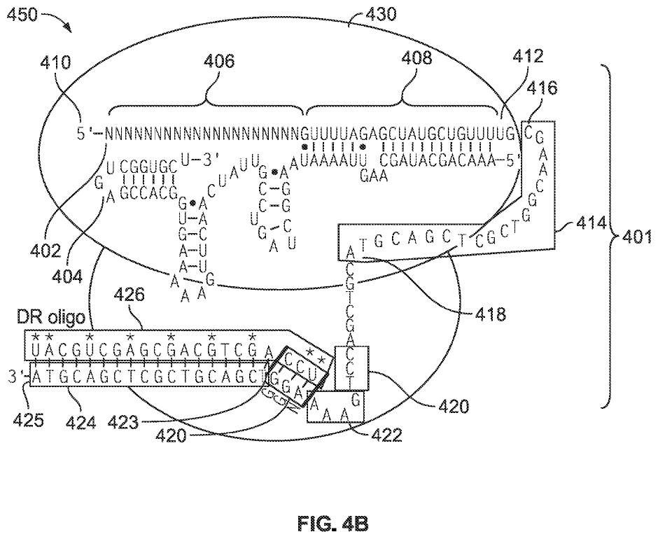

Referring now to FIG. 1, a CRISPR-Cas9 modulation system 100 is provided in the form of a composition comprising a nucleotide regulator 101 comprising cis-acting switchable nucleic acid-based regulators (cis-SNuBRs) comprising a CRISPR RNA (crRNA) 102, a trans-activated crRNA (tracr-RNA) 104, the crRNA comprising of a guide region 106, a tracrRNA pairing region 108, the crRNA 102 having a first end 110 and a second end 112, the second end 112 connected to a spacer 114, the spacer 114 having a first end 116 and a second end 118, a protospacer adjacent motif (PAM) mimic (PM) region 120 that begins at the end of the spacer 118, a hairpin region 122, and a toehold sequence 124 having a front end 123 connected to the PM region 120, and the toehold sequence having a back end 125. The nucleotide regulator 101 may additionally include a Cas protein 130 or a derepressor (not shown) (See FIG. 4B).

In various embodiments, the nucleotide regulator 101 may comprise a CRISPR repressor oligonucleotide. In various embodiments, the nucleotide regulator 101 may comprise a CRISPR repressor oligonucleotide and a derepressor oligonucleotide. The nucleotide regulator 101 may comprise a CRISPR repressor oligonucleotide as an artificial or synthetic oligonucleotide construct.

The nucleotide regulator 101 may comprise a cis-acting switchable nucleic acid-based regulators (cis-SNuBRs) including a crRNA 102, a tracr-RNA 104, the crRNA comprising of a guide region 106, a tracrRNA pairing region 108, the crRNA 102 having a first end 110 and a second end 112, the second end 112 connected to a spacer 114, the spacer 114 having a first end 116 and a second end 118, a protospacer adjacent motif (PAM) mimic (PM) region 120 beginning at the second end of the spacer 118, the PM region 120 including a hairpin structure 122 and ending at the front end 123 of the toehold sequence 124, with the toehold sequence having a back end 125.

The PM region 120 of the nucleotide regulator 101 may be capable of interacting with the protospacer adjacent motif (PAM)-interaction (PI) domain of a CRISPR-associated (Cas) protein, wherein the PM region 120 has a front 118 connected to the spacer 114 and back end 123 connected to the toehold sequence 124. The PM region includes a hairpin structure 122 which, when folded, inhibits the activity of the Cas protein 130.

The spacer 114 of the nucleotide regulator 101 includes a first end 116 which may be capable of attaching to the 3′ end 112 of the cRNA sequence 102 and a second end 112 attached to the to the polynucleotide capable of interacting with a PI domain of a Cas protein (e.g., the PM region 120). The spacer 114 polynucleotide may also be capable of attaching to the 3′ end of a typical crRNA sequence. In various embodiments, the first end 116 of the spacer 114 comprises a polynucleotide which may be operably connectable to the 3′ end 112 of the crRNA sequence 102. In other embodiments, the spacer 114 may include a nucleic acid sequence, or a chemical linker. In some embodiments, the spacer 114 is comprised of nucleotides and has a length of about 1 to about 22 nucleotides. In other embodiments, the spacer 114 has a length of about 1 to about 4 nucleotides. In other embodiments, the spacer 114 has a length of about 4 to about 7 nucleotides. In other embodiments, the spacer 114 has a length of about 7 to about 10 nucleotides. In other embodiments, the spacer 114 has a length of about 10 to about 13 nucleotides. In other embodiments, the spacer 114 has a length of about 13 to about 16 nucleotides. In other embodiments, the spacer 114 has a length of about 16 to about 19 nucleotides. In other embodiments, the spacer 114 has a length of about 19 to about 22 nucleotides. In other embodiments, the spacer 114 has a length of about 4 to about 10 nucleotides. In other embodiments, the spacer 114 has a length of about 16 to about 22 nucleotides.

In other embodiments, the nucleotide regulator 101 includes a spacer 114 with a length of about 0- about 50 nucleotides. In some embodiments, the spacer 114 has a length of about 1 nucleotide. In son embodiments, the spacer 114 has a length of about 4 nucleotides. In some embodiments, the spacer 114 has a length of about 7 nucleotides. In some embodiments, the spacer 114 has a length of about 10 nucleotides. In some embodiments, the spacer 114 has a length of about 13 nucleotides. In some embodiments, the spacer 114 has a length of about 16 nucleotides. In some embodiments, the spacer 114 has a length of about 19 nucleotides. In some embodiments, the spacer 114 has a length of about 22 nucleotides. In some embodiments, the nucleotide regulator 101 does not require a spacer 114.

In some embodiments, the spacer 114 may include a mixed nucleotide sequence. In other embodiments, the spacer 114 may include spacers composed of all A or all T nucleotides instead of a mixed sequence. In some embodiments, the spacer 114 may be comprised of a polymer.

In some embodiments, the nucleotide regulator 101 may comprise a linker molecule (not showi) instead of a spacer 114, the linker molecule attached to the PM region 120. In these embodiments, the linker molecule may be operably connected to the front end 118 of the PM region 120. The linker molecule may also be capable of attaching to the 3; end of a typical crRNA sequence 112. In various embodiments, the linker molecule may comprise polyethylene glycol (PEG). In other embodiments, the linker molecule may comprise poly-deoxythymidilic acid (poly-dT), or any other single stranded oligonucleotide with spacer functionality. In various embodiments, the linker molecule may comprise any biologically compatible polymer linker.

The nucleotide regulator 101 may also comprise a toehold polynucleotide sequence 124 attached to the PM region 120, wherein the toehold polynucleotide extends from the PM region 120 and comprises a front end 123 attached to the PM region 120 and a back end 125. The front end 123 of the toehold polynucleotide may be operably connected to the back end 123 of the PM region 120. In various embodiments, the toehold sequence 124 is designed to base-pair with another nucleic acid, which can be an exogenously supplied (e.g., a derepressor (not shown, See FIG. 4)) or an endogenously present sequence (such as a cellular RNA). In various embodiments, the base-pairing of the complementary derepressor, will compete with the hairpin sequence 122 of the PM region 120 which may release inhibition, thereby activating the Cas9 enzyme to bind substrate DNA (See FIG. 4B).

In some embodiments, the toehold 124 and spacer 114 sequence are comprised of broad chemistries, especially the spacer 114. In some embodiments, the toehold sequence 124 needs to bind to a complementary nucleic acid with high affinity so needs only to maintain Watson-Crick hybridization. The spacer 114 has only a spacing/linking function and can likely undergo the broadest chemical diversification and may not even need to be nucleic acid in nature, such as a polyethylene glycol (PEG) or polymer linker.

In various embodiments, the spacer 114, the PM region 120 with the hairpin 122, and the toehold sequence 124 are comprised of DNA.

The nucleotide regulator 101 may comprise a repressor oligonucleotide including 2′-deoxyribonucleotides only, 2′-deoxyribonucleotide analogs only, a mixture of 2′-deoxyribonucleotides and at least one 2′-deoxyribonucleotide analog, a mixture of 2′-deoxyribonucleotide analogs and at least one 2′-deoxyribonucleotide, or a mixture of 2′-deoxyribonucleotides and 2′-deoxyribonucleotide analogs.

The nucleotide regulator 101 may comprise a repressor oligonucleotide including ribonucleotides only, ribonucleotide analogs only, a mixture of ribonucleotides and at least one ribonucleotide analog, a mixture of ribonucleotide analogs and at least one ribonucleotide, or a mixture of ribonucleotides and ribonucleotide analogs.

The nucleotide regulator 101 may comprise a repressor oligonucleotide including at least two different types of nucleotides selected from the group consisting of: 2′-deoxyribonucleotide, 2′-deoxyribonucleotide analog, ribonucleotide, and ribonucleotide analog.

2′-deoxyribonucleotides described herein are generally understood to refer to 2′-deoxyribonucleic acid molecules that do not contain any chemical modifications. 2′-deoxyribonucleotide analogs described herein include any suitable chemically modified nucleotide that maintains the properties of a 2′-deoxyribonucleotide or mimics a 2′-deoxyribonucleotide. Some number of 2′-deoxyribonucleotide analogs are well-known to a person of skill in the art and can be used in certain embodiments of the present invention. In certain embodiments, examples include, but are not limited to, arabinonucleic acid (ANA), 2′-fluoro-arabinonucleic acid (2′-FANA), alpha-L locked nucleic acid (LNA), phosphorothioate deoxyribonucleic acid (PS-DNA), acyclic or unlocked nucleic acid (UNA), or any combination thereof.

Ribonucleotides as described herein are generally understood to refer to ribonucleic acid molecules that do not contain any chemical modifications. Ribonucleotide analogs as described herein include any suitable chemically modified nucleotide that maintains the properties of a ribonucleotide or mimics a ribonucleotide. Some number of ribonucleotide analogs are well-known to a person of skill in the art and can be used in certain embodiments of the disclosure. In some embodiments, examples include, but are not limited to, 2′-fluorinated ribonucleic acid (2′-FRNA) 2′,5-linked ribonucleic acid (2′, 5′-RNA), 3′,5′-linked ribonucleic acid (3′,5′-RNA), 2′-O-methyl ribonucleic acid (2′-OMe-RNA), 2′-methoxyethyl ribonucleic acid (2′-MOE-RNA), 2-fluorinated-4′-O-methyl ribonucleic acid (2′F,4′OMe-RNA), 2′,4′-di-O-methvl ribonucleic acid (2′4′-diOMe-RNA), 2′,4′ di-fluorinated ribonucleic acid (2′,4′-di-RN A), locked nucleic acid (LNA), and bicycle or bridged nucleic acid (BNA), or any combination thereof.

Some embodiments of the nucleotide regulator 101 including the CRISPR repressor oligonucleotides disclosed herein have conventional phosphodiester internucleotide linkages and are suitable for use in the disclosed embodiments, other internucleotide linkages are well-known to the skilled artisan and can be used in additional embodiments of the present disclosure. Thus, the present disclosure also provides polynucleotides that have one or more internucleotide linkage that is not a phosphodiester internucleotide linkage. In some embodiments, the nucleotide regulator 101 comprising a CRISPR repressor oligonucleotide includes at least one internucleotide linkage selected from the group consisting of phosphodiester, phosphorothioate, phosphotriester, phosphorodithioate, boranophosphate, Rp- and/or Sp-phosphorothioate, 3′ thioformacetal, methylene, amide, methylphosphonate, phosphoramidate and any combination thereof.

Some embodiments of the nucleotide regulator 101 including the repressor oligonucleotides described herein may be delivered using liposomes or a liposome-based carrier, Some embodiments of the nucleotide regulator 101 including the Cas protein described herein 130 may be delivered using liposomes or a liposome-based carrier. In various embodiments liposome preparation includes various designs without/with surface ligands for specific binding.

In some embodiments the nucleotide regulator 101 is delivered in vitro. In other embodiments, the nucleotide regulator 101 is delivered in vivo to a subject. In certain embodiments, the nucleotide regulator 101 is delivered to a human or animal. In some embodiments, the nucleotide regulator 101 transfected cell may be located in a subject. The subject may be an animal or a human. The subject may also be a plant.

In other embodiments, constructs of the present disclosure may take the form of an expression vector, which may be part of a plasmid or virus. In some embodiments, constructs of the present disclosure may contain a promoter operably linked to a transcribable nucleic acid molecule operably linked to a 3′ transcription termination nucleic acid molecule. In addition, constructs may include but are not limited to additional regulatory nucleic acid molecules from, e.g., the 3-untranslated region (3′ UTR). In certain embodiments, constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

Additionally, various embodiments of the nucleotide regulator 101 may comprise a CRISPR repressor oligonucleotide sequence selected from the group consisting of SEQ ID NO: 1 or a sequence at least 95% identical thereto or a functional fragment thereof, SEQ. ID. NO: 2 or a sequence at least 95% identical thereto or a functional fragment thereof, SEQ. ID. NO: 3 or a sequence at least 95% identical thereto or a functional fragment thereof, SEQ. ID. NO: 4 or a sequence at least 95% identical thereto or a functional fragment thereof, SEQ. ID. NO: 5 or a sequence at least 95% identical thereto or a functional fragment thereof, SEQ. ID. NO: 6 or a sequence at least 95% identical thereto or a functional fragment thereof, SEQ. ID. NO: 7 or a sequence at least 95% identical thereto or a functional fragment thereof, SEQ ID. NO. 8 or a sequence at least 95% identical thereto or a functional fragment thereof, SEQ. ID. NO. 9 or a sequence at least 95% identical thereto or a functional fragment thereof, SEQ. ID. NO: 10 or a sequence at least 95% identical thereto or a functional fragment thereof, SEQ. ID. NO: 11 or a sequence at least 05% identical thereto or a functional fragment thereof, SEQ. ID. NO: 12 or a sequence at least 95% identical thereto or a functional fragment thereof, SEQ. ID. NO: 13 or a sequence at least 95% identical thereto or a functional fragment thereof, SEQ. ID. NO: 14 or a sequence at least 95% identical thereto or a functional fragment thereof, SEQ. ID. NO: 15 or a sequence at least 95% identical thereto or a functional fragment thereof, SEQ. ID. NO: 16 or a sequence at least 95% identical thereto or a functional fragment thereof, SEQ. ID. NO: 17 or a sequence at least 95% identical thereto or a functional fragment thereof, SEQ. ID. NO: 18 or a sequence at least 95% identical thereto or a functional fragment thereof, SEQ. ID. NO: 19 or a sequence at least 95% identical thereto or a functional fragment thereof, and SEQ. ID. NO: 29 or a sequence at least 95% identical thereto or a functional fragment thereof.

Additionally, various embodiments of the nucleotide regulator 101 may comprise a CRISPR repressor oligonucleotide with a spacer sequence selected from the group consisting of: SEQ. ID. NO: 21 or a sequence at least 95% identical thereto or a functional fragment thereof, SEQ. ID, NO: 22 or a sequence at least 95% identical thereto or a functional fragment thereof, SEQ. ID. NO: 23 or a sequence at least 95% identical thereto or a functional fragment thereof. SEQ. ID. NO: 24 or a sequence at least 95% identical thereto or a functional fragment thereof, SEQ. ID. NO: 25 or a sequence at least 95% identical thereto or a functional fragment thereof, SEQ. ID. NO: 26 or a sequence at least 95% identical thereto or a functional fragment thereof, SEQ. ID. NO: 27 or a sequence at least 95% identical thereto or a functional fragment thereof, SEQ. ID. NO: 28 or a sequence at least 95% identical thereto or a functional fragment thereof, thereof.

The Cas protein 130 may be selected from the group consisting of Cas9, Cas12 (Cpf1), dead Cas9 (dCas9). In other embodiments, the Cas protein may be any other suitable Cas protein from a CRISPR system. In some embodiments, the Cas protein is Cas9. While the most common source of Cas9 protein is from Streptococcus pyogenes, other sources of Cas protein are well- known to those of skill in the art and may be used in some embodiments of the disclosure provided. Examples of other sources of Cas protein include, but are not limited to, Staphylococcus aureus, Campylobacter jejuni, Neisseria meningitides, Francisilla novicida, Streptococcus thermophiles, Geobacillus stearothermophilus, and numerous other bacterial species.

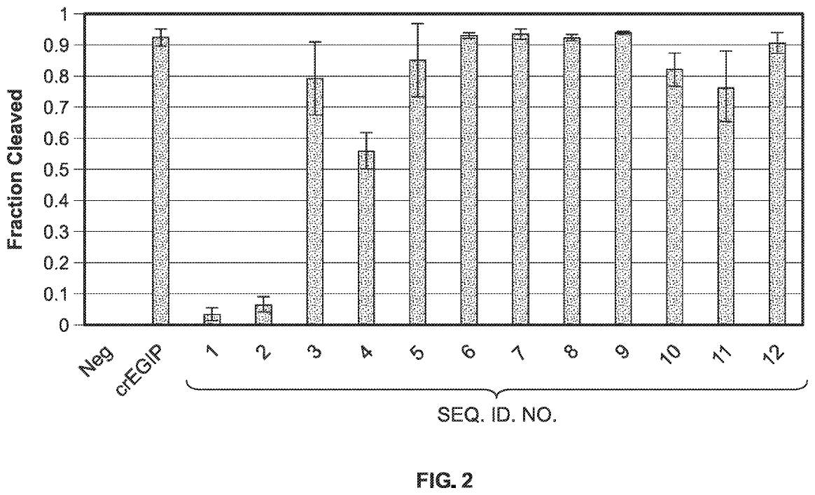

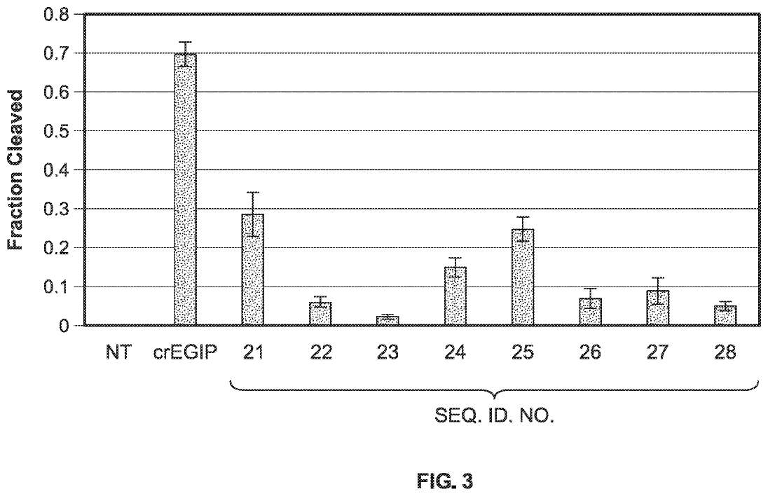

Referring now to FIG. 2, which depicts a graph showing the inhibition of in vitro cleavage of target substrate DNA by Cas9 (Cas9 cleavage activity) for the various DNA oligonucleotides SEQ. ID No. 1-12.

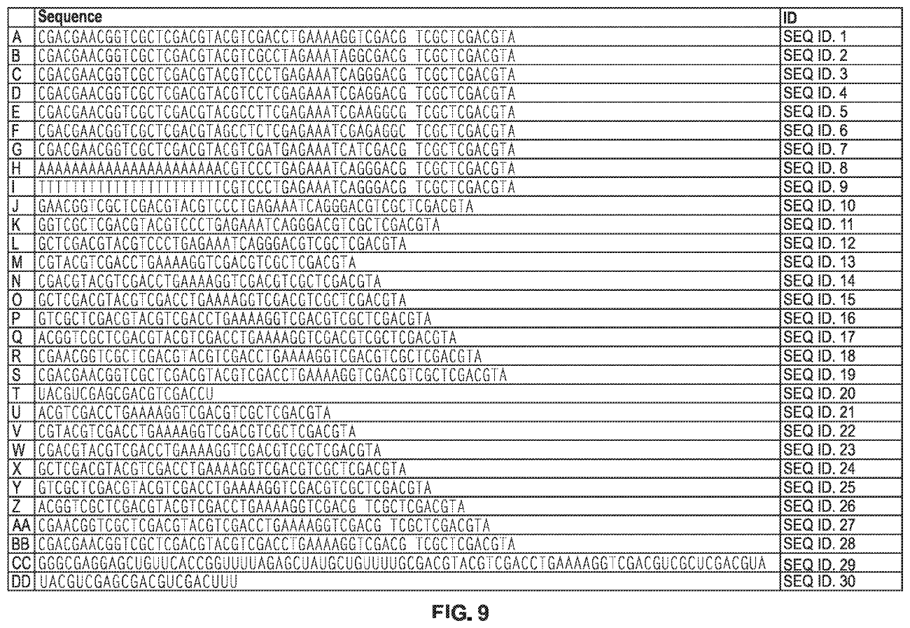

Twelve exemplary DNA oligonucleotides (See FIG. 9 for SEQ. ID. sequences) with 5′ phosphates were splint-ligated to the 3′ end of a typical crRNA sequence. SEQ. ID. NOs 1-6 contained varying placements of the AGG PAM sequence in the PM module 120, starting directly adjacent to the 3′ end of the GAAA tetraloop (SEQ. ID. NO. 1) (See FIG. 1, hairpin structure 122) and then systematically sliding 1 nucleotide away (SEQ. ID NOs 2-12). SEQ. ID. NO. 7 contained no PAM sequence and served as a negative control. SEQ. ID. NOs 8-9 comprised alternative spacers composed of all A or all T nucleotides not a mixed sequence. SEQ. ID. NOs. 10-12, comprised alternative spacer lengths of 18, 14, and 10 nucleotides, respectively, instead of 22 nucleotides. crEGIP as described in Berkau et al. 2019 and incorporated herein was used as a control. In one embodiment, the strongest inhibition of in vitro cleavage of target substrate DNA by Cas9 was observed when the PAM sequence was directly adjacent to the GAAA tetraloop (See FIG. 2 SEQ. ID. No. 1).

In various embodiments, a cell transfected with the nucleotide regulator 101 including the CRISPR repressor oligonucleotide may have inhibited Cas9 cleavage activity compared to a control cell lacking the CRISPR repressor oligonucleotide.

In some embodiments, administration of the repressor oligonucleotide may result in inhibited Cas9 cleavage activity which may comprise at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% decrease in Cas9 cleavage activity compared to the control cell lacking the repressor oligonucleotide.

In some embodiments, the inhibited Cas9 cleavage activity may comprise at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% decrease in Cas9 cleavage activity compared to the control cell lacking the CRISPR repressor oligonucleotide.

Referring now to FIG. 3, which depicts the inhibition of in vitro cleavage of target substrate DNA by Cas9 (Cas9 cleavage activity) for nucleotide regulators with the various DNA oligonucleotides SEQ. ID. NOs 21-28;

In various embodiments spacer 114 length can be between about 0- about 50 nucleotides. In various embodiments, the spacer 114 length of the repressor oligomer 101 may be systematically increased from about 1 nucleotide to about 22 nucleotides in length.

Various embodiments with spacer 114 variations are exemplified by SEQ. ID. NOs 21-28 (See FIG. 9 for SEQ. ID sequences). Inhibition of in vitro cleavage was achieved with the multiple embodiments of the spacer 114 variation SEQ. ID. NOs. 21-28. crEGIP as described in Berkau et al. 2019 and incorporated herein was used as a control.

SEQ. ID. NO. 23, with a spacer 114 of 7 nucleotides in length, possessed the greatest degree of inhibition of Cas9 which decreased upon increase in spacer 114 length (FIG. 3 SEQ. ID. Nos 24-25) but inhibition of Cas9 increased with longer spacer 114 length (FIG. 3 SEQ. ID. NO 28). In some embodiments of the nucleotide regulator 101 including a repressor oligonucleotide 101, the spacer 114 may comprise a possible periodicity in the optimal spacer length.

Referring now to FIGS. 4A and 4B, a CRISPR-Cas9 modulation system mechanism comprising a repressor 400 and a derepressor 450 is provided in the form of a composition comprising a nucleotide regulator 401 comprising cis-acting switchable nucleic acid-based regulators (cis-SNuBRs) comprising a CRISPR RNA (crRNA) 402, a trans-activated crRNA (tracr-RNA) 404, the crRNA comprising of a guide region 406, a tracrRNA pairing region 408, the crRNA 402 having a first end 410 and a second end 412, the second end 412 connected to a spacer 414, the spacer 414 having a first end 416 and a second end 418, a protospacer adjacent motif (PAM) mimic (PM) region 420 that begins at the end of the spacer 418, a hairpin region 422, and a toehold sequence 424 having a front end 423 connected to the PM region 420, and the toehold sequence having a back end 425. The nucleotide regulator 401 may additionally include a Cas protein 430 or a derepressor 426.

In various embodiments, the nucleotide regulator 401 may comprise a CRISPR repressor oligonucleotide. In various embodiments, the nucleotide regulator 401 may comprise a CRISPR repressor oligonucleotide and a derepressor oligonucleotide. The nucleotide regulator 401 may comprise a CRISPR repressor oligonucleotide as an artificial or synthetic oligonucleotide construct. The nucleotide regulator 401 may comprise a derepressor 426 oligonucleotide as an artificial or synthetic oligonucleotide construct.

The nucleotide regulator 401 may comprise a cis-acting switchable nucleic acid-based regulators (cis-SNuBRs) including a crRNA 402, a tracr-RNA 404, the crRNA comprising of a guide region 406, a tracrRNA pairing region 408, the crRNA 402 having a first end 410 and a second end 412, the second end 412 connected to a spacer 414, the spacer 414 having a first end 416 and a second end 418, a protospacer adjacent motif (PAM) mimic (PM) region 420 beginning at the second end of the spacer 418, the PM region 420 including a hairpin structure 422 and ending at the front end 423 of the toehold sequence 424, with the toehold sequence having a back end 425.

The PM region 420 of the nucleotide regulator 401 may be capable of interacting with the protospacer adjacent motif (PAM)-interaction (PI) domain of a CRISPR-associated (Cas) protein, wherein the PM region 420 has a front 418 connected to the spacer 414 and back end 423 connected to the toehold sequence 424. The PM region includes a hairpin structure 422 which, when folded, inhibits the activity of the Cas protein 430 (FIG. 4A).

The spacer 414 of the nucleotide regulator 401 includes a first end 416 which may be capable of attaching to the 3′ end 412 of the cRNA sequence 402 and a second end 412 attached to the to the polynucleotide capable of interacting with a PI domain of a Cas protein (e.g., the PM region 420). The spacer 414 polynucleotide may also be capable of attaching to the 3′ end of a typical crRNA sequence. In various embodiments, the first end 416 of the spacer 414 comprises a polynucleotide which may be operably connectable to the 3′ end 412 of the crRNA sequence 402. In other embodiments, the spacer 414 may include a nucleic acid sequence, or a chemical linker. In some embodiments, the spacer 414 is comprised of nucleotides and has a length of about 1 to about 22 nucleotides. In other embodiments, the spacer 414 has a length of about 1 to about 4 nucleotides. In other embodiments, the spacer 414 has a length of about 4 to about 7 nucleotides. In other embodiments, the spacer 414 has a length of about 7 to about 10 nucleotides. In other embodiments, the spacer 414 has a length of about 10 to about 13 nucleotides. In other embodiments, the spacer 414 has a length of about 13 to about 16 nucleotides. In other embodiments, the spacer 414 has a length of about 16 to about 19 nucleotides. In other embodiments, the spacer 414 has a length of about 19 to about 22 nucleotides. In other embodiments, the spacer 414 has a length of about 4 to about 10 nucleotides. In other embodiments, the spacer 414 has a length of about 16 to about 22 nucleotides.

In other embodiments, the nucleotide regulator 401 includes a spacer 414 with a length of about 0- about 50 nucleotides. In some embodiments, the spacer 414 has a length of about 1 nucleotide. In some embodiments, the spacer 414 has a length of about 4 nucleotides. In some embodiments, the spacer 414 has a length of about 7 nucleotides. In some embodiments, the spacer 414 has a length of about 10 nucleotides. In some embodiments, the spacer 414 has a length of about 13 nucleotides. In some embodiments, the spacer 414 has a length of about 16 nucleotides. In some embodiments, the spacer 414 has a length of about 19 nucleotides. In some embodiments, the spacer 414 has a length of about 22 nucleotides. In some embodiments, the nucleotide regulator 401 does not require a spacer 414.

In some embodiments, the spacer 414 may include a mixed nucleotide sequence. In other embodiments, the spacer 414 may include spacers composed of all A or all T nucleotides instead of a mixed sequence. In some embodiments, the spacer 414 may be comprised of a polymer.

In some embodiments, the nucleotide regulator 401 may comprise a linker molecule (not shown) instead of a spacer 414, the linker molecule attached to the PM region 420. In these embodiments, the linker molecule may be operably connected to the front end 418 of the PM region 420. The linker molecule may also be capable of attaching to the 3; end of a typical crRNA sequence 412. In various embodiments, the linker molecule may comprise polyethylene glycol (PEG). In other embodiments, the linker molecule may comprise poly-deoxythymidilic acid (poly-dT), or any other single stranded oligonucleotide with spacer functionality. In various embodiments, the linker molecule may comprise any biologically compatible polymer linker.

The nucleotide regulator 401 may also comprise a toehold poly nucleotide sequence 424 attached to the PM region 420, wherein the toehold polynucleotide extends from the PM region 420 and comprises a front end 423 attached to the PM region 420 and a back end 425. The front end 423 of the toehold polynucleotide may be operably connected to the back end 423 of the PM region 420. In various embodiments, the toehold sequence 424 is designed to base-pair with another nucleic acid, which can be an exogenously supplied (e.g., a derepressor 426) or an endogenously present sequence (such as a cellular RNA). In various embodiments, the base-pairing of the complementary derepressor 426, will compete with the hairpin sequence 422 of the PM region 420 which may release inhibition, thereby activating the Cas9 430 enzyme to bind substrate DNA 450.

In some embodiments, the toehold 424 and spacer 414 sequence are comprised of broad chemistries, especially the spacer 414. In some embodiments, the toehold sequence 424 needs to bind to a complementary nucleic acid with high affinity so needs only to maintain Watson-Crick hybridization. The spacer 414 has only a spacing/linking function and can likely undergo the broadest chemical diversification and may not even need to be nucleic acid in nature, such as a polyethylene glycol (PEG) or polymer linker.

In various embodiments, the spacer 414, the PM region 420 with the hairpin 422, and the toehold sequence 424 are comprised of DNA.

In various embodiments, the nucleotide regulator may comprise a repressor oligonucleotide sequence of mG*mG*rGrCrGrArGrGrArGrCrUrGrUrUrCrArCrCrGrGrUrUrUrUrArGrArGrCrUrArUrG rCrUrGrUrUmUmU*mG*C*G*A*C*G*T*A*mCGTCGACCTG*A*A*AAGGTCGACGm UmCmGmCmUmCmGmAmCmG*mU*mA wherein N indicates nucleotide, mN indicates 2′-O-methyl modified. * indicates a phosphorothioate bond, rN indicates RNA nucleotide.

The nucleotide regulator 401 may comprise a repressor oligonucleotide including 2′-deoxyribonucleotides only, 2′-deoxyribonucleotide analogs only, a mixture of 2′-deoxyribonucleotides and at least one 2′-deoxyribonucleotide analog, a mixture of 2′-deoxyribonucleotide analogs and at least one 2′-deoxyribonucleotide, or a mixture of 2′-deoxyribonucleotides and 2′-deoxyribonucleotide analogs.

The nucleotide regulator 401 may comprise a repressor oligonucleotide including ribonucleotides only, ribonucleotide analogs only, a mixture of ribonucleotides and at least one ribonucleotide analog, a mixture of ribonucleotide analogs and at least one ribonucleotide, or a mixture of ribonucleotides and ribonucleotide analogs.

The nucleotide regulator 401 may comprise a repressor oligonucleotide including at least two different types of nucleotides selected from the group consisting of 2′-deoxyribonucleotide, 2′-deoxyribonucleotide analog, ribonucleotide, and ribonucleotide analog. [0089]2′-deoxyribinucleotides described herein are generally understood to refer to 2′-deoxyribonucleic acid molecules that do not contain any chemical modifications. 2′-deoxyribonucleotide analogs described herein include any suitable chemically modified nucleotide that maintains the properties of a 2′-deoxyribinucleotide or mimics a 2′-deoxyribonucleotide. Some number of 2′-deoxyribonucleotide analogs are well-known to a person of skill in the art and can be used in certain embodiments of the present invention. In certain embodiments, examples include, but are not limited to, arabinonucleic acid (ANA). 2′-fluoro-arabinonucleic acid (2′-FANA), alpha-L locked nucleic acid (LNA), phosphorothioate deoxyribonucleic acid (PS-DNA), acyclic or unlocked nucleic acid (UNA), or any combination thereof.

Ribonucleotides as described herein are generally understood to refer to ribonucleic acid molecules that do not contain any chemical modifications. Ribonucleotide analogs as described herein include any suitable chemically modified nucleotide that maintains the properties of a ribonucleotide or mimics a ribonucleotide. Some number of ribonucleotide analogs are well-known to a person of skill in the art and can be used in certain embodiments of the disclosure. In some embodiments, examples include, but are not limited to, 2′-fluorinated ribonucleic acid (2′-FRNA), 2′,5′-linked ribonucleic acid (2′,5′-RNA), 3′,5′-linked ribonucleic acid (3′,5′-RNA), 2′-O-methyl ribonucleic acid (2′-OMe-RNA), 2′-methoxyethyl ribonucleic acid (2′-MOE-RNA), 2′-fluorinated-4′-O-methyl ribonucleic acid (2′F,4′OMe-RNA), 2′,4′-di-O-methyl ribonucleic acid (2′,4′-diOMe-RNA), 2′,4′ di-fluorinated ribonucleic acid (2′,4′-di-RNA), locked nucleic acid (LNA), and bicycle or bridged nucleic acid (BNA), or any combination thereof.

Some embodiments of the nucleotide regulator 401 including the CRISPR repressor oligonucleotides disclosed herein have conventional phosphodiester internucleotide linkages and are suitable for use in the disclosed embodiments, other internucleotide linkages are well-known to the skilled artisan and can be used in additional embodiments of the present disclosure. Thus, the present disclosure also provides polynucleotides that have one or more internucleotide linkage that is not a phosphodiester internucleotide linkage. In some embodiments, the nucleotide regulator 401 comprising a CRISPR repressor oligonucleotide includes at least one internucleotide linkage selected from the group consisting of phosphodiester, phosphorothioate, phosphotriester, phosphorodithioate, boranophosphate, Rp- and/or Sp-phosphorothioate, 3′ thioformacetal, methylene, amide, methylphosphonate, phosphoramidate and any combination thereof.

Some embodiments of the nucleotide regulator 401 including the repressor oligonucleotides described herein may be delivered using liposomes or a liposome-based carrier. Some embodiments of the nucleotide regulator 401 including the Cas protein described herein 130 may be delivered using liposomes or a liposome-based carrier. In various embodiments liposome preparation includes various designs without/with surface ligands for specific binding.

In some embodiments the nucleotide regulator 401 is delivered in vitro. In other embodiments, the nucleotide regulator 401 is delivered in vivo to a subject. In certain embodiments, the nucleotide regulator 401 is delivered to a human or animal. In some embodiments, the nucleotide regulator 401 transfected cell may be located in a subject. The subject may be an animal or a human. The subject may also be a plant.

In other embodiments, constructs of the present disclosure may take the form of an expression vector, which may be part of a plasmid or virus. In some embodiments, constructs of the present disclosure may contain a promoter operably linked to a transcribable nucleic acid molecule operably linked to a 3′ transcription termination nucleic acid molecule. In addition, constructs may include but are not limited to additional regulatory nucleic acid molecules from, e.g., the 3′-untranslated region (3′ UTR). In certain embodiments, constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

Additionally, various embodiments of the nucleotide regulator 401 may comprise a CRISPR repressor oligonucleotide sequence selected from the group consisting of SEQ ID NO: 1 or a sequence at least 95% identical thereto or a functional fragment thereof, SEQ. ID. NO: 2 or a sequence at least 95% identical thereto or a functional fragment thereof, SEQ. ID. NO: 3 or a sequence at least 95% identical thereto or a functional fragment thereof, SEQ. ID. NO: 4 or a sequence at least 95% identical thereto or a functional fragment thereof, SEQ. ID. NO: 5 or a sequence at least 95% identical thereto or a functional fragment thereof, SEQ. ID. NO: 6 or a sequence at least 95% identical thereto or a functional fragment thereof, SEQ. ID. NO: 7 or a sequence at least 95% identical thereto or a functional fragment thereof, SEQ. ID. NO: 8 or a sequence at least 95% identical thereto or a functional fragment thereof, SEQ. ID. NO: 9 or a sequence at least 95% identical thereto or a functional fragment thereof, SEQ. ID. NO: 10 or a sequence at least 95% identical thereto or a functional fragment thereof, SEQ. ID. NO: 11 or a sequence at least 95% identical thereto or a functional fragment thereof, SEQ. ID. NO: 12 or a sequence at least 95% identical thereto or a functional fragment thereof, SEQ. ID. NO: 13 or a sequence at least 95% identical thereto or a functional fragment thereof, SEQ. ID. NO: 14 or a sequence at least 95% identical thereto or a functional fragment thereof, SEQ. ID. NO: 15 or a sequence at least 95% identical thereto or a functional fragment thereof, SEQ. ID. NO: 16 or a sequence at least 95% identical thereto or a functional fragment thereof, SEQ. ID. NO: 17 or a sequence at least 95% identical thereto or a functional fragment thereof, SEQ. ID. NO: 18 or a sequence at least 95% identical thereto or a functional fragment thereof, SEQ. ID. NO: 19 or a sequence at least 95% identical thereto or a functional fragment thereof, and SEQ. ID. NO: 29 or a sequence at least 95% identical thereto or a functional fragment thereof.

Additionally, various embodiments of the nucleotide regulator 401 may comprise a CRISPR repressor oligonucleotide with a spacer sequence selected from the group consisting of: SEQ. ID. NO: 21 or a sequence at least 95% identical thereto or a functional fragment thereof, SEQ. ID. NO: 22 or a sequence at least 95% identical thereto or a functional fragment thereof, SEQ. ID. NO: 23 or a sequence at least 95% identical thereto or a functional fragment thereof, SEQ. ID. NO: 24 or a sequence at least 95% identical thereto or a functional fragment thereof, SEQ. ID. NO: 25 or a sequence at least 95% identical thereto or a functional fragment thereof, SEQ. ID. NO: 26 or a sequence at least 95% identical thereto or a functional fragment thereof, SEQ. ID. NO: 27 or a sequence at least 95% identical thereto or a functional fragment thereof, SEQ. ID. NO: 28 or a sequence at least 95% identical thereto or a functional fragment thereof, thereof.

The Cas protein 430 may be selected from the group consisting of Cas9, Cas12 (Cpf1), dead Cas9 (dCas9). In other embodiments, the Cas protein may be any other suitable Cas protein from a CRISPR system. In some embodiments, the Cas protein is Cas9. While the most common source of Cas9 protein is from Streptococcus pyogenes, other sources of Cas protein are well- known to those of skill in the art and may be used in some embodiments of the disclosure provided. Examples of other sources of Cas protein include, but are not limited to, Staphylococcus aureus, Campylobacter jejuni, Neisseria meningitides, Francisilla novicida, Streptococcus thermophiles, Geobacillus stearothermophilus, and numerous other bacterial species.

In some embodiments, the derepressor oligonucleotide 426 may be an artificial or synthetic oligonucleotide construct. In various embodiments, the derepressor oligonucleotide 426 may comprise a polynucleotide capable of interacting with the toehold polynucleotide. In other embodiments, the derepressor oligonucleotide 426 may comprise a polynucleotide that is complementary to the toehold polynucleotide. Alternatively, the derepressor oligonucleotide 426 may comprise a polynucleotide that is partially complementary to the toehold polynucleotide (FIG. 4B).

In some embodiments, the derepressor 426 oligonucleotide may comprise a polynucleotide that is complementary to the polynucleotide capable of interacting with a PI domain of a Cas protein 430. Alternatively, the derepressor 426 oligonucleotide may comprise a polynucleotide that is partially complementary to the polynucleotide capable of interacting with a PI domain of a Cas protein 430. The derepressor 426 oligonucleotide may comprise a polynucleotide that is complementary to the toehold 424 polynucleotide and partially complementary to the polynucleotide capable of interacting with a PI domain of a Cas protein 430. In other embodiments, the derepressor oligonucleotide 426 may comprise a polynucleotide that is partially complementary to the toehold 424 polynucleotide and partially complementary to the polynucleotide capable of interacting with a PI domain of a Cas protein 430 (FIG. 4B).

The derepressor oligonucleotide 426 may comprise modified nucleotides such as methyl modified nucleotides. In FIG. 4B, the described embodiment includes a derepressor 426 which contains 2′-O-methyl modified nucleotides as indicated by the asterisk symbol.

In some embodiments, the derepressor oligonucleotide may comprise of a sequence of mUmACGmUCGmAGCmGACmGTCmGACmCmU where N indicates nucleotide and mN indicates 2′-O-methyl modified).

In some embodiments, the derepressor may comprise of eUeAeCeG+UeCeGeA+G eCeGeA+CeGeUeC+GeAeCeU+UeU wherein N indicates nucleotide, eN indicates 2′-methoxyethyl (MOE) modified, and +N indicates locked nucleic acid (LNA) modified.

The derepressor oligonucleotide 426 may comprise 2′-deoxyribonucleotides only, 2′-deoxyribonucleotide analogs only, a mixture of 2′-deoxyribonucleotides and at least one 2′-deoxyribonucleotide analog, a mixture of 2′-deoxyribonucleotide analogs and at least one 2′-deoxyribonucleotide, or a mixture of 2′-deoxyribonucleotides and 2′-deoxyribonucleotide analogs.

The derepressor oligonucleotide may comprise ribonucleotides only, ribonucleotide analogs only, a mixture of ribonucleotides and at least one ribonucleotide analog, a mixture of ribonucleotide analogs and at least one ribonucleotide, or a mixture of ribonucleotides and ribonucleotide analogs.

The derepressor oligonucleotide 426 may comprise at least two different types of nucleotides selected from the group consisting of: 2′-deoxyribonucleotide, 2′-deoxyribonucleotide analog, ribonucleotide, and ribonucleotide analog.

Alternatively, the derepressor oligonucleotide 426 may comprise SEQ ID NO: 20 or a sequence at least 95% identical thereto or a functional fragment thereof.

Alternatively, the derepressor oligonucleotide 426 may comprise SEQ ID NO: 30 or a sequence at least 95% identical thereto or a functional fragment thereof.

In various embodiments, a cell transfected with the nucleotide regulator 401 repressor oligonucleotide may have inhibited Cas9 cleavage activity compared to a control cell lacking the repressor oligonucleotide. In various embodiments, a cell transfected with both the CRISPR repressor oligonucleotide and the derepressor oligonucleotide may have increased Cas9 cleavage activity compared to a control cell transfected with the CRISPR repressor oligonucleotide but lacking the derepressor oligonucleotide.

In some embodiments, administration of the repressor oligonucleotide may result in inhibited Cas9 cleavage activity which may comprise at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% decrease in Cas9 cleavage activity compared to the control cell lacking the repressor oligonucleotide.

In various embodiments, the administration of the derepressor 426 oligonucleotide after the administration of the repressor may rescue the Cas9 cleavage activity by at least about 1%, at least about 2%, at least about 3%, at least about 4%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% increase in Cas9 cleavage activity compared to the control cell transfected with the CRISPR repressor oligonucleotide but lacking the derepressor oligonucleotide.

In some embodiments, the inhibited Cas9 cleavage activity may comprise at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% decrease in Cas9 cleavage activity compared to the control cell lacking the CRISPR repressor oligonucleotide.

In various embodiments, the administration of the derepressor 426 oligonucleotide after the administration of the repressor may rescue the Cas9 cleavage activity by at least 1%, at least 2%, at least 3%, at least 4%, at least 5%, at least 6%, at least 7%, at least 8%, at least about 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% increase in Cas9 cleavage activity compared to the control cell transfected with the CRISPR repressor oligonucleotide but lacking the derepressor oligonucleotide.

In various embodiments, the nucleotide regulator 401 Cis-SNuBRs make use of a dual-guide CRISPR-Cas9 system where the tracrRNA 404 is maintained but the crRNA 402 is modified to possess a protospacer adjacent motif (PAM) mimic (PM) module at its 3′ end 412. The PM module 420 is a nucleic acid that folds into a minimal duplex structure that contains a typical PAM sequence and mimics a fragment of the natural DNA substrate of the Cas protein 430 (e.g., Cas9). The PM module 420 is directly connected to the crRNA by a spacer 414, which can be a nucleic acid sequence or a chemical linker. The PM module 420 is positioned so as to bind to the PAM-interacting (PI) domain of Cas9, thereby inhibiting its DNA substrate binding and catalytic activities. Extending from the other end of the PM module is a ‘toehold’ 424 sequence. The toehold 424 is designed to base-pair with another nucleic acid, which can be an exogenously supplied (such as a ‘derepressor’ 426 oligo) or endogenously present sequence (such as a cellular RNA) (FIGS. 1 and 4). Base-pairing of the complementary derepressor 426 oligonucleotides, for example, will compete with PM module's 420 hairpin structure 422 and release inhibition, thereby activating the Cas9 enzyme 430 to bind substrate DNA (FIG. 4).

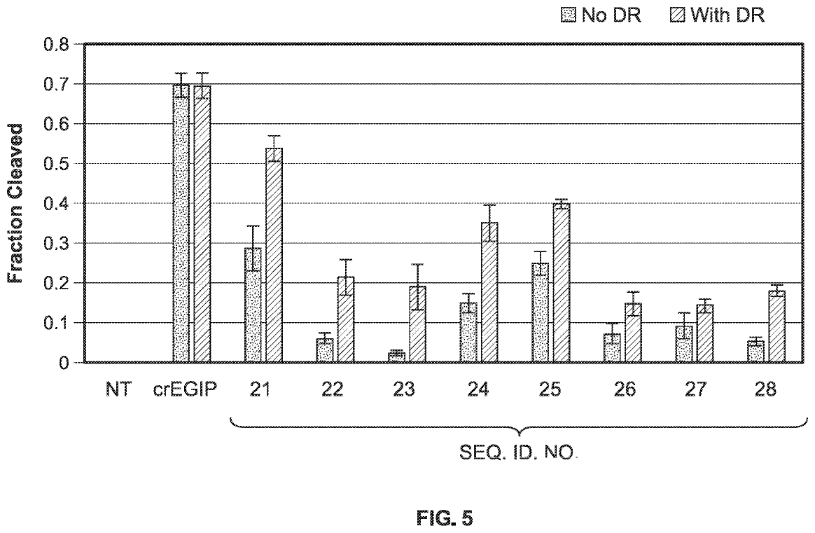

Referring now to FIG. 5, which depicts the in vitro cleavage of Cas9 in the presence of the nucleotide regulator 101 or 401 comprising the repressor spacer variant oligonucleotides and the derepressor 426;

In various embodiments, the derepressor 426 oligonucleotide may have the ability to derepress the inhibition caused by the varying spacer oligonucleotide repressor designs. In one embodiment, a derepressor (DR) oligonucleotide sequence (see FIG. 5) was added into an in vitro cleavage reaction after the addition of a repressor oligonucleotide (SEQ. ID. NOs 21-28) as described herein. crEGIP as described in Berkau et al. 2019 and incorporated herein was used as a control. SEQ. ID. NOs 21-28 reduced the fraction of the target gene cleaved by Cas9 reduced Cas9 activity) while addition of a derepressor showed observable derepression (rescued Cas 9 activity) and increased the in vitro cleavage fraction of the target gene, for all spacer oligonucleotides (FIG. 5).

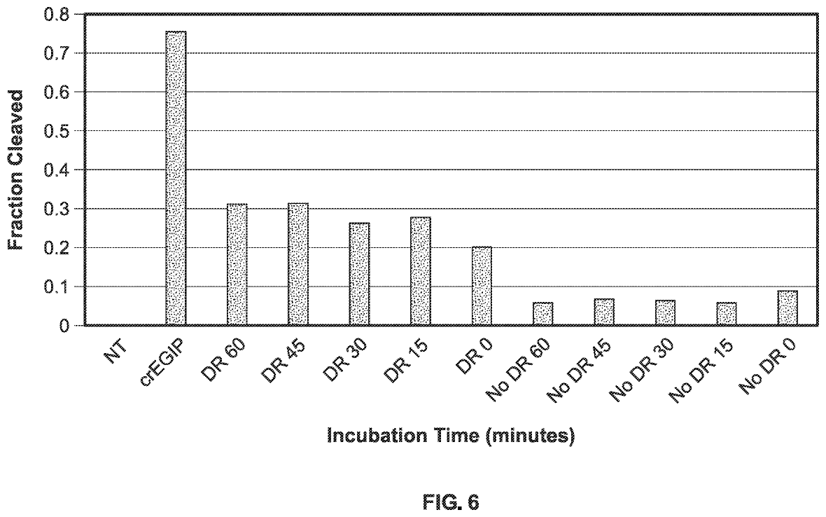

Referring now to FIG. 6, which discloses the in vitro cleavage activity of Cas9 in in the presence of the repressor oligonucleotides and the derepressor (DR) with varying derepressor incubation times (in minutes). Pre-incubation with the DR oligonucleotide 426 may alter the degree of Cas9 cleavage activity. In one embodiment, pre-incubation for 15 min improved Cas9 activity, likely by improved derepression, but did not continue to improve Cas9 activity linearly with longer incubations up to 60 min (FIG. 6). crEGIP as described in Berkau et al. 2019 and incorporated herein was used as a control.

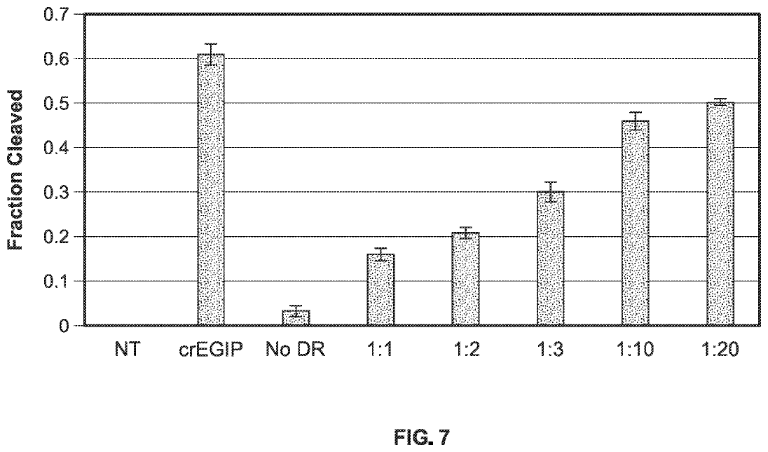

Referring now to FIG. 7, which shows the in vitro cleavage activity of Cas9 in in the presence of the repressor oligonucleotides and the derepressor with varying concentrations (molar ratios of 1:1, 1:2, 1:5, 1:10, and 1:20). The derepression may be driven by competitive binding of the DR oligonucleotide 426. In some embodiments wherein the molar ratio of DR to repressor is altered by adding increasing concentrations of the DR oligonucleotide, Cas9 activity may increase proportionally with the DR concentration. The Cas9 activity plateaued beyond a 10-fold molar excess of the DR oligonucleotide over the SEQ. ID. NO. 23 oligonucleotide fused to the crRNA (FIG. 7. See FIG. 9 for description of sequences). crEGIP as described in Berkau et al. 2019 and incorporated herein was used as a control.

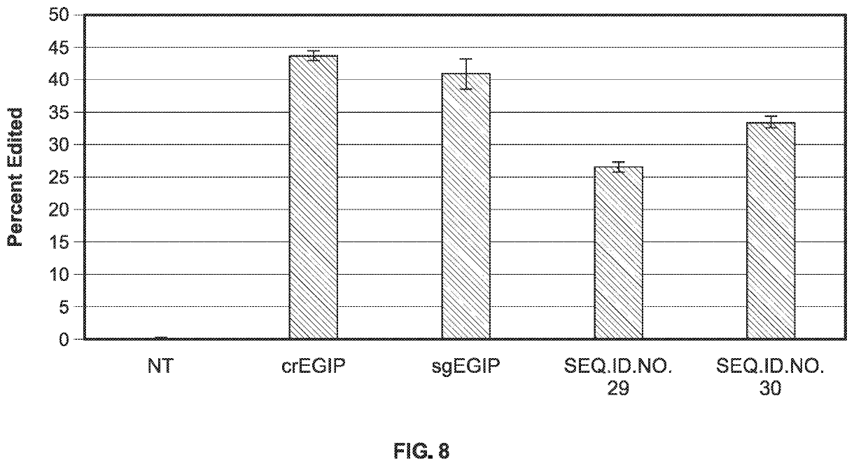

Referring now to FIG. 8, which describes the precent edited gene target by Cas9 in the presence of the repressor oligonucleotide SEQ. ID. NO. 29 and the derepressor SEQ. ID. NO. 30. (See FIG. 9 for description of sequences). In various embodiments, the nucleotide regulator can comprise of a repressor and a derepressor wherein one or both oligonucleotides have modified nucleotide chemistries including but not limited to the following modifications: 2′-O-methyl modifications, phosphorothioate bonds, RNA nucleotide instead of DNA nucleotide, 2′-methoxyethyl (MOE) modifications, and locked nucleic acid (LNA) modifications. In one embodiment. SEQ. ID. NO. 29 was capable of reducing the percent edited gene target of Cas9 (inhibiting Cas9 activity) while SEQ. ID. NO. 30 was able to partially rescue the percent edited gene target (regaining Cas9 activity). crEGIP and sqEGIP as described in Berkau et al. 2019 and incorporated herein were used as controls.

Referring now to FIG. 9, which discloses the sequences of oligonucleotides used in the detailed description and various embodiments of the disclosure. In the sequences as presented, FIG. 9 provides nucleotide sequences as used in various embodiments as described herein.

Further embodiments of the disclosure described herein include modification of sequence and structure variants of the nucleotide regulator, such as testing and variance of the sequence and structure of each feature (guide region, tracrRNA-pairing region, spacer, PM module, and toehold) of the cis-SNuBR. In other embodiments, the cis-SNuBR design may also be installed on a single guide RNA (sgRNA) scaffold of CRISPR-Cas9, such as by chemically tethering the spacer. PM module, and toehold features to the tetraloop of the sgRNA. Alternatively, the same features could be linked to the end of the tracrRNA, such as the 5′ end which is closest to the Cas9 PI domain.

Additional embodiments include modification of the disclosed nucleotide regulators comprising cis-SNuBRs to include diverse chemical modification schemes to protect the cis-SNuBR molecule from nucleases, adjust stability and Watson-Crick hybridization properties, and tune deliverability to cells or target tissues in an organism. In various embodiments, the cis-SNuBRs may be chemically modified in the crRNA portion using RNA-like 2′-fluoro or 2′-O-methyl, while the PM module will likely need to maintain DNA-like structure for PT domain interaction. In some embodiments, 2′-fluoro arabinonucleic acid (FANA) may be used as a modifier. FANA is a DNA- like nucleotide analogue, which possesses rigid DNA-like sugar pucker and folded (B-form) structure. In other embodiments, cis-SNuBRs may be modified via backbone phosphodiester modifications which are likely to be broadly tolerated, such as phosphorothioate. In some embodiments, the toehold and spacer sequences are likely to be able to tolerate very broad chemistries, especially the spacer. The toehold needs to bind to a complementary nucleic acid with high affinity so needs only to maintain Watson-Crick hybridization. The spacer has only a spacing/linking function and can likely undergo the broadest chemical diversification and may not even need to be nucleic acid in nature, such as a polyethylene glycol (PEG) or polymer linker.

The embodiments and compositions described herein may be generalizable to nearly all CRISPR-Cas systems and may function through a mechanism that involves occluding the natural DNA (PAM) binding by the Cas protein. In other embodiments, chemical modification of the cis-SNuBR and crRNA fusion may yield highly stable molecules with near 100% inhibition and high switchability to the active, substrate binding- competent state of the Cas protein. In other embodiments, use of a derepressor oligonucleotide may also enable tissue or target specific activation of Cas9. In some embodiments, chemical modifications and conjugates to the depressor nucleotide that target certain tissues or organs or allow formulation-free cellular uptake are highly conceivable. As described herein, the derepressor oligonucleotide disclosed is composed of alternating DNA and 2′-O-methyl nucleotides.

In various embodiments, modifications to the structure and chemistry of the cis-SNuBR system may yield more effective or therapeutically active molecules, however they may not be expected to significantly change the mechanism of action. In some embodiments, other methods to switch from inactive to active states of Cas9 may involve different chemical or biological approaches, such as photocleavable or enzymatically labile spacers or a peptide-linked toehold. These approaches would act by either competing with the PM module structure and its recognition by the PI domain or else releasing the PM module altogether to uncouple it from the Cas9-RNA complex.

The following definitions and methods are provided to better define the present invention and to guide those of ordinary skill in the art in the practice of the present invention. Unless otherwise noted, terms are to be understood according to conventional usage by those of ordinary skill in the relevant art.

The term “construct” is understood to refer to any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked.

An “expression vector”, “vector”, “vector construct”, “expression construct”, “plasmid”, or “recombinant DNA construct” is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. Conversely, a homologous DNA sequence is a DNA sequence that is naturally associated with a host cell into which it is introduced. [00128]“Operably linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions, and methods for preparing and using constructs and host cells are well known to one skilled in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. When used in conjunction with the word “comprising” or other open language in the claims, the words “a” and “an” denote “one or more,” unless specifically noted. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that all of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and this can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1 SpCas9 Protein Preparation

A plasmid, pET-Cas9-NLS-6×His, was transformed into Rosetta 2 (DE3) cells which contain chloramphenicol resistance. Next, the cultures were expanded, and protein expression was induced using 0.4 mM IPTG for 24 hours at 18° C. Cells were pelleted and resuspended in chilled binding buffer (20 mM Tris HCl pH 7.5, 250 mM NaCl, 5 mM Imidazole) supplemented with 1 mM PMSF to inhibit protease activity. Then, the cells were lysed by French press at 1100 psi and the lysate was clarified through centrifugation.

The enzyme was purified by Cobalt-CMA Column Chromatography. Next, the resin was equilibrated with binding buffer and the soluble layer of the clarified lysate was added. This was mixed and incubated at 4° C. for 1 hour to ensure binding of the 6×-His-tagged Cas9 protein to the resin. The resin-bound protein was washed with increasing concentrations of NaCl solution and eluted in 15 mL of elution buffer (20 mM Tris HCl, pH 7.5, 250 mM NaCl, 130 mM imidazole). Next, the eluted protein was concentrated using a 10,000 MWCO concentrator tube and buffer was exchanged using 2× Storage Buffer (40 mM Tris HCl, pH 7.5, 300 mM KCl, 1 mM EDTA, 2 mM DTT) to minimize the imidazole concentration. Finally, the concentration of the protein was measured using absorbance at 280 nm and the calculated molar extinction coefficient of 120,575 M−1 cm-1. One volume of 100% glycerol was added to the sample which was then stored at −20° C.

### Example 2 Splint Ligations of crRNA to Cis-SNuBR Oligonucleotides

The designed cis-SNuBR oligonucleotides were purchased from Integrated DNA Technologies (IDT). Then, those oligonucleotides are ligated to a crRNA guide by IDT. This was done by synthesizing a custom splint to join the crRNA guide to the cis-SNuBR oligonucleotides. The crRNA guide, splint, and cis-SNuBR oligonucleotides were mixed with a slight excess of splint to ensure that all molecules were bound. RNA Resuspension Buffer (5 mM Tris pH 7.2, 0.1 mM EDTA) was used to bring the total volume of the reaction to 200 μL. The samples were boiled at 95° C. for 5 minutes and annealed by slow cooling to room temperature.

Next, for the ligation reaction, the crowding reagents 9 M Betaine and 30% w/v PEG8000 were added to aid in ligation efficiency. T4 DNA Ligase was added at a 0.03 μM final concentration along with T4 DNA Ligase Buffer and SUPERase-In™ (an RNase Inhibitor). Then, the reaction was incubated at 37° C. for 6 hours and then phenol/chloroform extracted.

Next, the extracted product was gel purified on a 15% Denaturing PAGE gel that ran at 40 mA for ˜3 hours. The bands of interest were cut out and rotated in elution buffer (20 mM Tris pH 7.5, 0.3 M Sodium Acetate, 5 mM EDTA, 0.1% SDS) at 4° C. overnight. Next, the eluate was collected and filtered in paper spin cups (Pierce™ spin cups) to remove leftover gel, while more elution buffer was added to the gel and rotated at room temperature for 1 hour. This step was repeated a second time and the entire eluate was filtered again. Next, purified nucleic acids were ethanol precipitated, dried, and resuspended in RNA Resuspension Buffer. Finally, concentration of the cis-SNuBRs were determined using absorbance at 260 nm and a calculated molar extinction coefficient.

### Example 3. In Vitro Cleavage Activity Assays

A Cas9 RNP was assembled using 0.0625 μM SpCas9 and 0.25 μM tracrRNA via incubation at 37° C. for 10 minutes with 1× cleavage buffer and 0.1 mg/mL tRNA. During incubation, the cis-SNuBRs at a final concentration of 0.3 μM final were spotted into tubes along with 160 ng of the target. Then, for experiments testing the derepressor oligo, this was also spotted into the tubes at a final concentration of 0.3 μM (1:1 ratio with cis-SNuBR) and typically incubated at 37° C. for 30 minutes to allow interaction between the derepressor and cis-SNuBR prior to the cleavage reaction. Next, the reaction was initiated by adding the ribonucleoprotein (RNP) complex to the cis-SNuBR/derepressor/target mix with a final reaction volume of 40 μL. This was incubated at 37° C. for 1 hour. In timepoint assays, the derepressor oligo was not pre-incubated with the cis-SNuBR and instead added as the reaction progressed.

Then, to end the reaction, 2% LiClO4 in acetone was added to the reaction and precipitated for 1-24 hours at −20° C. Next, the sample was pelleted by centrifugation, washed, dried, resuspended with distilled and deionized water (ddH_{2O}) and treated with RNase A and Proteinase.

Then. 6× Purple Loading Dye (New England Biolabs) was added and the samples were run on 1.5% TBE-buffered agarose gels that were stained ethidium bromide. Next, gels were placed on a UV Imager for visualization and measurement of band intensity. Afterwards, quantification was performed using ImageJ where the fraction of target DNA cleaved was measured. Finally, the quantification was calculated by taking the intensity of the band for the cleavage product (cut) and dividing it by the sum of the intensities of the cleavage product and the uncleaved (uncut) substrate.

1. A composition comprising a nucleotide regulator, wherein the nucleotide regulator is an oligonucleotide sequence at least 95% identical to a sequence selected from the group consisting of SEQ. ID. NOs. 1-6 and 8-19 or a functional fragment thereof.

2. The composition of claim 1, further comprising an oligonucleotide sequence comprising a linker or a spacer of about 0- about 50 nucleotides in length.

3. The composition of claim 2, further comprising a Cas protein.

4. The composition of claim 3, further comprising a tracrRNA and wherein the Cas protein comprises Cas9, or dead Cas9 (dCas9).

5. The composition of claim 4, further comprising a derepressor.

6. The composition of claim 5, further comprising a derepressor oligonucleotide.

7. The composition of claim 6, wherein the composition comprises a derepressor to nucleotide regulator ratio from about 1:1 to about 1:20.

8. The composition of claim 7, wherein the derepressor oligonucleotide is an oligonucleotide sequence at least 95% identical to SEQ ID 20 or a functional fragment thereof.

9. A method for selectively altering expression of at least one gene product comprising administering the composition of claim 1 to a subject.

10. The method of claim 9, wherein the composition further comprises an oligonucleotide sequence comprising a linker or a spacer of about 0- about 50 nucleotides in length.

11. The method of claim 10, wherein the composition further comprises a Cas protein.

12. The method of claim 10, the composition further comprising a tracrRNA and wherein the Cas protein comprises Cas9, or dead Cas9 (dCas9).

13. The method of claim 12, wherein the composition further comprises a derepressor.

14. The method of claim 13, wherein the composition further comprises a derepressor oligonucleotide.

15. The method of claim 14, wherein the composition has a derepressor to nucleotide regulator ratio from about 1:1 to about 1:20.

16. The method of claim 15 wherein the derepressor is at least 95% identical to SEQ ID 20 or a functional fragment thereof.

17. A method of selectively modulating the activity of a Cas protein comprising administering a composition comprising a repressor and a derepressor to a subject, wherein the repressor competes with or disrupts the PM module structure of the Cas protein, and wherein the derepressor reverses the disruption of the repressor.

18. The method of claim 17, wherein the repressor comprises a nucleotide regulator, wherein the nucleotide regulator is at least 95% identical to a sequence selected from the group consisting of SEQ ID NOs. 1-6 and 8-19 or a functional fragment thereof.

19. The method of claim 18, wherein the derepressor comprises an oligonucleotide sequence at least 95% identical to SEQ ID 20 or a functional fragment thereof.

20. The method of claim 17, wherein the repressor and derepressor are included in a liposome.
